(19) United States
(12) Patent Application Publication
(10) Pub. No.: US 2021/0353536 A1
(43) Pub. Date: Nov. 18, 2021

(54) Applying Fluoride for Treating or Preventing Viral Infection Including SARS-CoV-2

(71) Applicant: Reynolds Delgado, Houston, TX (US)

(72) Inventor: Reynolds Delgado, Houston, TX (US)

(21) Appl. No.: 16/882,815

(22) Filed: May 26, 2020

Related U.S. Application Data
(60) Provisional application No. 63/023,948, filed on May 13, 2020.

(51) Int. Cl. A61K 9/00; A61K 9/12; A61K 9/107; A61K 33/16; A61K 9/50

(52) U.S. Cl. CPC A61K 9/0078; A61K 9/12; A61K 9/1075; A61K 47/26; A61K 9/0053; A61K 9/50; A61K 33/16

(57) ABSTRACT

Disclosed is inhaling or spraying sodium fluoride (NaF) or other fluoride salts, e.g., stannous fluoride, potassium fluoride, calcium fluoride, or magnesium fluoride, or other fluoride compounds, e.g., sodium monofluoro phosphate, in solution or in emulsion (carried in, e.g., a lipid phase) or where the solution or emulsion is encapsulated in microparticles into the mouth region and/or nasal cavities for treatment or prophylaxis of viral infections, including SARS-CoV-2 and other corona viruses, influenza, parainfluenza and rhinoviruses (which are the primary agents responsible for common colds) and other human and animal respiratory pathogenic viruses.

### BACKGROUND

When one gets a cold or flu, it is usually first felt in the back of the throat, the sinuses and the lungs. Usually it is not felt in the mouth area; which is counter-intuitive, considering that the mouth or nose is usually the first point of contact with the virus, and further considering that the mouth has the same mucosa as the throat, sinuses and bronchi. One might speculate that the mouth remains protected because most people, on average twice daily, coat the oral cavity with sodium fluoride during teeth brushing. Sodium fluoride, and other fluoride salts or fluoride-containing compounds, may protect against infection or inactivate viruses, which may be why wide experience is that initially, one doesn't get viral infections in the oral cavity. Sodium fluoride and sodium monofluoro phosphate are FDA approved and often present in toothpaste, mouthwash and even in drinking water, and are widely considered extremely safe.

A system to coat the initial infection sites in the nasal passages, sinuses, pharynx and bronchi with fluoride, is warranted to try to protect against or treat viral infections, including Corona viruses like SARS-CoV-2.

### SUMMARY

The invention relates to inhaling or spraying (using, e.g., the familiar squeeze applicator for nasal administration of solutions), sodium fluoride (NaF) or other fluoride salts, e.g., stannous fluoride, potassium fluoride, calcium fluoride, or magnesium fluoride, or other fluoride compounds, e.g., sodium monofluoro phosphate, in solution or in emulsion (carried in, e.g., a lipid phase) or where the solution or emulsion is encapsulated in microparticles (see U.S. Pat. No. 8,569,028, incorporated by reference) into the mouth region and/or nasal cavities for treatment or prophylaxis of viral infections, including coronaviruses such as SARS-CoV-2, influenza, parainfluenza, rhinoviruses (which are the primary agents responsible for common colds) and other respiratory human pathogenic viruses. In another embodiment, the fluoride salts or compounds in solution or in emulsion could be inhaled or sprayed under pressure (using e.g., a conventional metered dose inhaler used for asthma treatment, or another atomizer, nebulizer or aerosolizer) into the bronchial airways and then exhaled through the nose or mouth to coat all those regions with fluoride.

In another embodiment, fluoride salts or compounds in solution, emulsion or encapsulated in microparticles is administered using a device which preferably has three nozzles, one for each nostril and one for the mouth. One such embodiment would squeeze spray or pressure spray (using e.g., an aerosolizer, atomizer or nebulizer) the fluoride salts or compounds in solution, emulsion or encapsulated in particles into the mouth, then activate a de-pressurization to withdraw fluoride through nostrils. Alternatively, a squeeze-spray device could spray through an outlet, or could pressure spray the fluoride composition into both nasal passage and into the mouth, at the same time.

In another embodiment, fluoride salts or compounds in solution could be administered to a patient on a ventilator by adding it to the ventilator circuit, as is common for administration of bronchodilators. See e.g., C. Guerin et al., “Inhaled Bronchodilator Administration During Mechanical Ventilation: How to Optimize It, and for Which Clinical Benefit?” J Aerosol Med Pulm Drug Deliv 2008 21(1) 85-96. This would be helpful in treating pneumonia or bronchitis which are virus-related.

Fluoride salts or compounds in solution or in emulsion could also be mixed with dispersants or emulsifiers, or other carrier molecules or preservatives, for inhalation or spray administration. Suitable emulsifiers include benzalkonium chloride and Polysorbate 80 (polyoxyethylene (20) sorbitan monoleate). Suitable preservatives include phenethyl alcohol, sodium benzoate, methyl paraben, and ethyl paraben. Other carriers and formulants may be added as needed, including formulants in inhaler formulations like microcrystalline cellulose, carboxymethyl cellulose and dextrose. If any of the components in any formulation above are too large for delivery by an inhaler, they could likely be administered with another device, like a conventional squeeze applicator.

Another embodiment has the fluoride salts or compounds in solution or in emulsion administered after or together with a mucolytic, e.g., acetylcysteine, to reduce the mucous layer covering cells in the nose and airway mucosa, which might inhibit efficacy.

Compositions which increase the fluoride residence time, such as gels or gelling agents, or other carriers, thickeners, surfactants or humectants could also be included. Examples of pharmaceutically acceptable carriers include glycerin, white wax, petrolatum, stearyl alcohol, propylene glycol, sodium lauryl sulfate, propyl paraben, methyl paraben, and peppermint oil. In one embodiment, compounds which increase the fluoride residence time are applied after administration of the fluoride salts or compounds in solution or in emulsion. Such compounds need not be spray-applied.

### BRIEF DESCRIPTION OF THE DRAWINGS

FIG. 1 is a perspective view of the control valve and outlet portions of an inhaler device.

FIG. 2 is a frontal elevational view of the control valve and outlets in FIG. 1.

FIG. 3 is an exploded view of a control valve and outlets with a propellant canister with its holder.

FIG. 4 is the assembled view of the components in FIG. 3.

FIG. 5 is an exploded view of a control valve and outlets with an adapter and an electric driver unit suitable for both propulsion and de-pressurization.

FIG. 6 is a perspective view of the components in FIG. 5 partially assembled.

FIG. 7 is a perspective view of the components in FIG. 5 fully assembled.

### DETAILED DESCRIPTION

In one embodiment, a squeeze applicator (which is compressed by the user to provide a spray at the outlet(s)) or a propellant-powered or electrically powered aerosolizer, atomizer or nebulizer provides the fluoride salts or compounds in emulsion or encapsulated in particles (the “active ingredients”) into the bronchi, pharynx and sinuses, by application into the mouth and/or either or both nostrils. Preferably, the emulsion or the particles provide differently sized emulsion droplets or particles, such that larger droplets or particles will deposit in sinuses and the smaller ones will travel to the pharynx and bronchi. This differential can be controlled by adjusting the formulation composition or by using differently sized or shaped nozzles in the outlets. Whether or not differently-sized particles are present, following application, the user can exhale through the nose and mouth to better coat all those regions with fluoride; or an electrically-powered unit could perform de-pressurization to achieve such a coating.

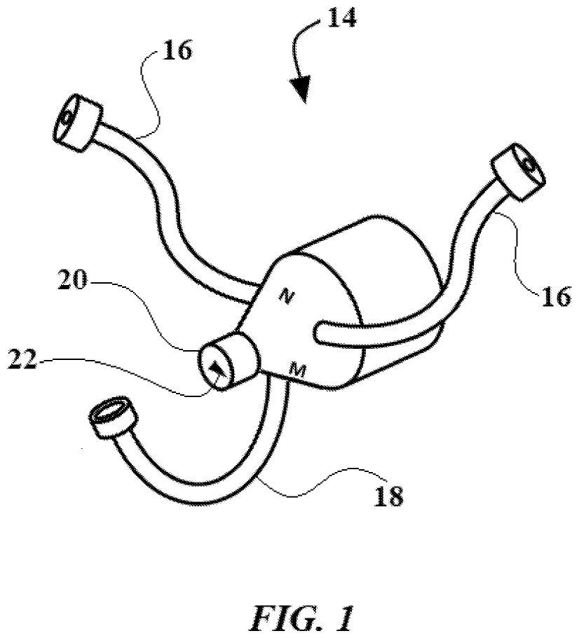

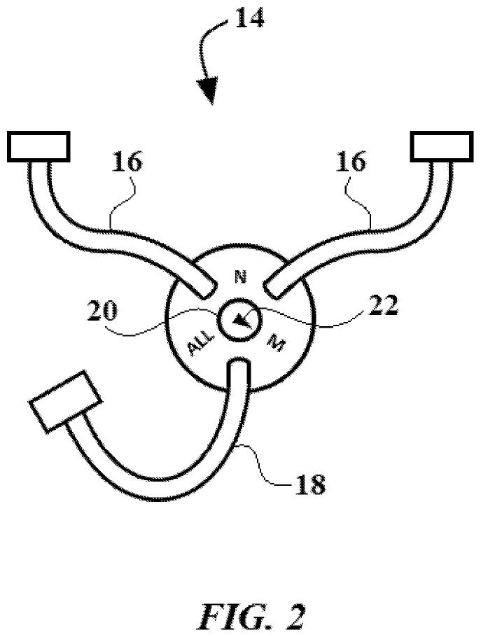

Referring to FIG. 1, an applicator valve 14 with outlets 16 for the nostrils and 18 for the mouth is shown. As best seen in FIG. 2, control 20 allows selective opening and closing of the outlets 16 and 18, so administration can be through all three outlets, or nostrils only or mouth only, as displayed on indicator 22.

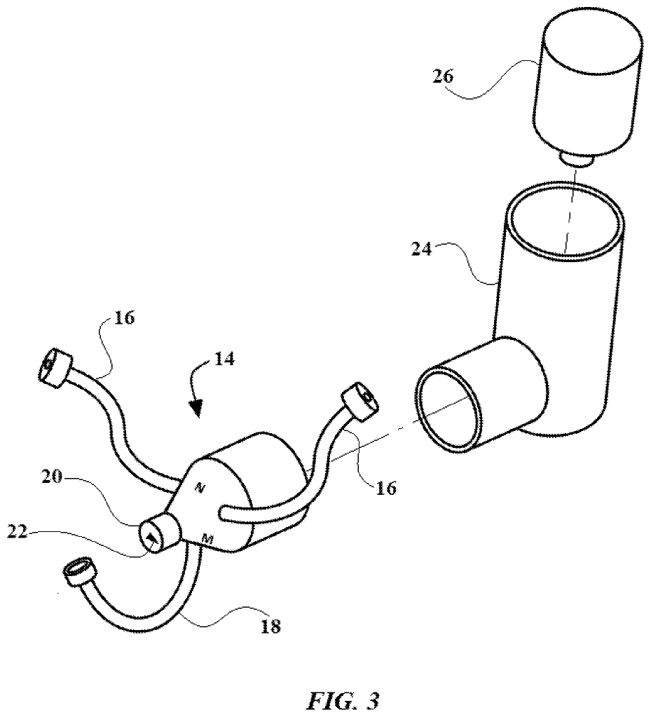

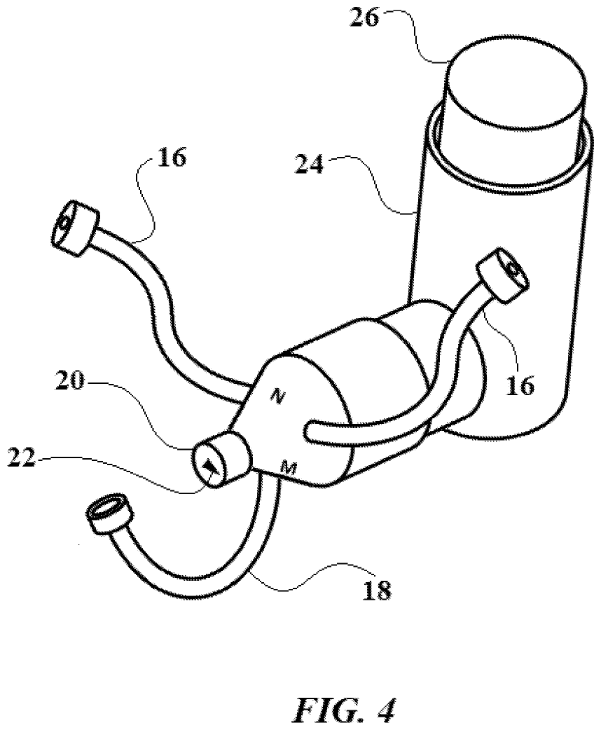

FIGS. 3 and 4 depict a metered dose inhaler apparatus with a propulsion canister 26 and holder 24 which are attached to the applicator valve 14 of FIGS. 1 and 2, for delivery of the active ingredients. In this embodiment, the system is a metered dose inhaler and canister 26 houses pressurized chlorofluorocarbon (CFC) propellants, as is a common delivery means for bronchodilators for asthma. HFA-134a, 1,1,1,2-tetrafluoroethane, is another suitable propellant for inhalers, which is non-ozone depleting and is reported to be quickly cleared from the body after inhalation.

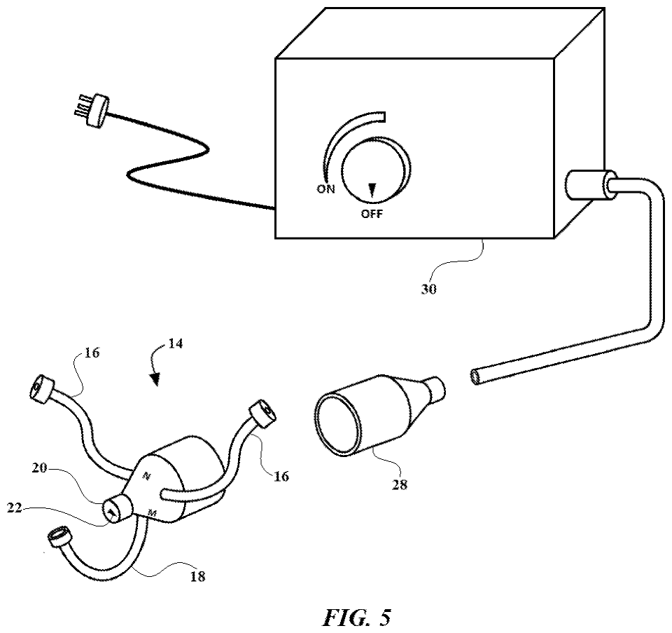

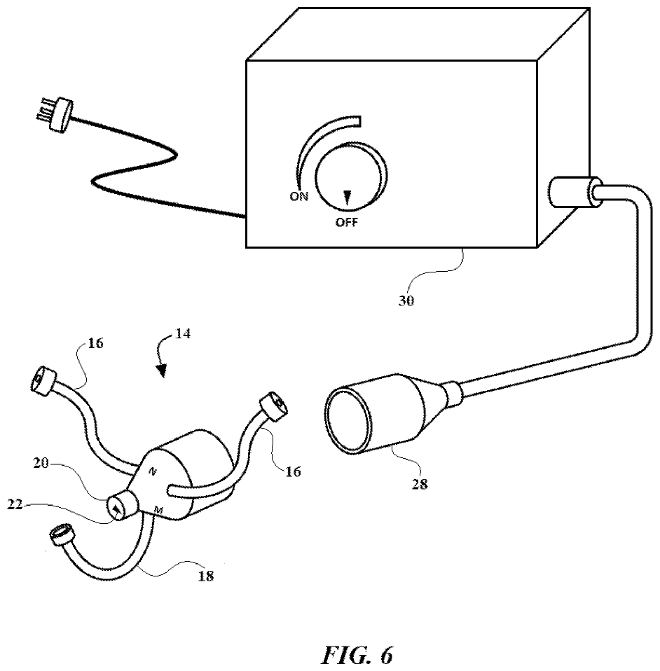

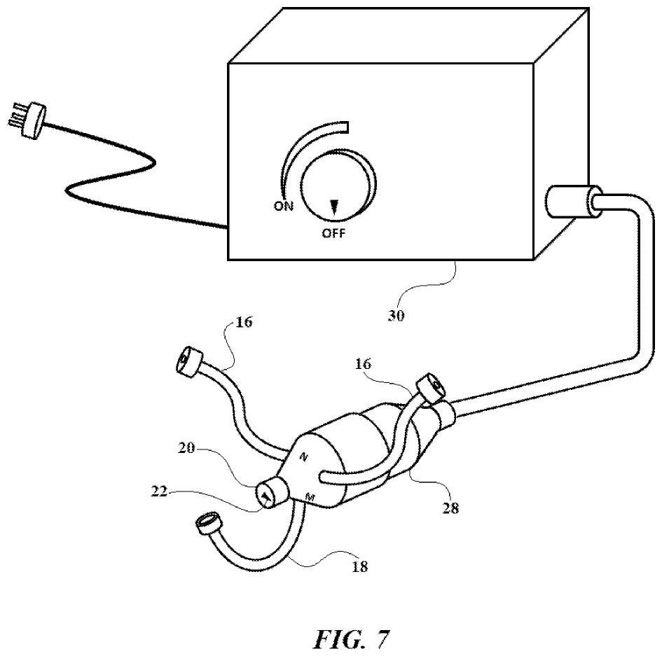

FIGS. 5 to 7 depict a jet nebulizer system which is less portable, but could also deliver the active ingredients. A jet nebulizer converts the active ingredients into a fine mist that the patient can inhale through a face mask, or it can be applied with the applicator valve 14 and related components, as shown. An electrically powered driver 30 feeds the nebulized active ingredients through an adaptor 28, which provides a sealed transition from the outlet tube for the driver 30 to the applicator valve 14. This system can also provide a de-pressurization after application, to withdraw the composition through the mouse and nose.

The required dosage of the active ingredients to effectively prevent or treat viral infections can be readily determined by routine experimentation with cellular or animal models, and then extrapolation. For example, after determining the effective concentration in a mouse model, the amount administered to a human subject can be determined by increasing the quantity administered to the mouse by the ratio of the weight of a human over a mouse. Routine experimentation on human subjects can also used to determine the effective dosage, especially if combined with use of artificial intelligence or machine learning to follow cellular and animal data and trends, to determine a suitable extrapolated dosage for human subjects; and thereafter, AI and machine learning can be used to track subjects in clinical trials to determine safety and efficacy in the treatment or prevention of viral infections.

The embodiments, components, steps, features, objects, benefits, and advantages which have been discussed are merely illustrative, and not limiting. All articles, patents, patent applications, and other publications that have been cited in this disclosure are incorporated herein by reference. The terms “comprises,” “comprising,” “including” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type. The invention is defined only in the claims which follow and includes all equivalents of the elements in the claims.

1. A formulation comprising an atomized, aerosolized or nebulized solution or emulsion of sodium fluoride, stannous fluoride, potassium fluoride, calcium fluoride, magnesium fluoride, or sodium monofluoro phosphate.

2. The formulation of claim 1 wherein the solution or emulsion is encapsulated in microparticles.

3. The formulation of claim 1 including an emulsifier which is benzalkonium chloride or polyoxyethylene (20) sorbitan monoleate.

4. A delivery system comprising a solution or emulsion of sodium fluoride, potassium fluoride, calcium fluoride, magnesium fluoride, or sodium monofluoro phosphate and a component to atomize, aerosolize or nebulize the solution or emulsion.

5. The delivery system of claim 4 wherein the solution or emulsion is encapsulated in microparticles.

6. The delivery system of claim 4 including an emulsifier which is benzalkonium chloride or polyoxyethylene (20) sorbitan monoleate.

7. The delivery system of claim 4 wherein the component is a compressible applicator, a pressurized canister or an electrically powered driver.

8. The delivery system of claim 7 wherein the pressurized canister houses pressurized chlorofluorocarbon or 1,1,1,2-tetrafluoroethane.

9. The delivery system of claim 4 having three outlets.

10. The delivery system of claim 9 wherein two of the outlets are designed to fit into a subject's nostrils and one outlet is designed to be held in the subject's mouth.

11. A method of preventing or treating SARS-CoV-2 viral infections comprising administering an atomized, aerosolized or nebulized solution or emulsion of fluoride to the nasal passages, sinuses, pharynx or bronchi of a subject in an amount effective to prevent or treat the SARS-CoV-2 viral infection.

12. (canceled)

13. The method of claim 11 wherein the fluoride is sodium fluoride, stannous fluoride, potassium fluoride, calcium fluoride, magnesium fluoride, or sodium monofluoro phosphate.

14. The method of claim 11 wherein the emulsion includes an emulsifier which is benzalkonium chloride or polyoxyethylene (20) sorbitan monoleate.

15. (canceled)

16. The method of claim 11 wherein the solution or emulsion is sprayed into the mouth and into each nostril simultaneously.

17. The method of claim 11 wherein the solution or emulsion is administered into the mouth and into one or both nostrils and the size of the droplets in the solution or emulsion administered to the nostrils is smaller than those administered to the mouth.

18. The method of claim 11 wherein the solution or emulsion is administered into the mouth and the subject is instructed to exhale through the nose and mouth following administration.

19. (canceled)

20. (canceled)
